(19) United States
(12) Patent Application Publication
(10) Pub. No.: US 2013/0110078 A1
(43) Pub. Date: May 2, 2013

(54) Vaccination Method Using Microneedle Arrays

(71) Applicants: Anne C. Moore, Cork (IE); Frances Pearson, W.Yorkshire (GB); Adrian V.S. Hill, Oxford (GB); Abina M. Crean, Cork (IE); Conor O'Mahony, Cork (IE); Marie G. McGrath, Cork (IE); John B. Carey, Mayo (IE)

(72) Inventors: Anne C. Moore, Cork (IE); Frances Pearson, W.Yorkshire (GB); Adrian V.S. Hill, Oxford (GB); Abina M. Crean, Cork (IE); Conor O'Mahony, Cork (IE); Marie G. McGrath, Cork (IE); John B. Carey, Mayo (IE)

(21) Appl. No.: 13/643,964

(22) PCT Filed: Apr. 28, 2011
(86) PCT No.: PCT/GB11/00664

§ 371 Date: Jan. 4, 2013

Related U.S. Application Data
(60) Provisional application No. 61/329,438, filed on Apr. 29, 2010.

(30) Foreign Application Priority Data
Apr. 29, 2010 (GB) 1007207.2

(51) Int. Cl. A61M 37/00

(52) U.S. Cl. CPC A61M 37/0015
USPC 604/506; 604/173

(57) ABSTRACT

The present invention provides vaccination methods using microneedle arrays which, when applied to the skin of a subject, create a defined total pore volume. Arrays may be chosen of varying total microneedle volumes (small, intermediate or large) for use in immunisation regimes to induce different T-cell and antibody responses in the subject. The present invention also provides kits for use in such vaccination method(s) comprising the appropriate microneedle array(s).

### FIELD OF THE INVENTION

The present invention relates to vaccination kits and methods which use microneedle array devices for percutaneous delivery. In particular, it relates to vaccination kits and methods using particular designs of microneedle arrays which induce different T-cell and antibody responses in the vaccinated subject.

### BACKGROUND TO THE INVENTION

Vaccines may be administered through various routes of delivery, including oral, nasal, intramuscular (IM) or intradermal (ID). The route of administration can significantly impact on the magnitude and phenotype of this response. While vaccination represents the primary public health measure to combat infectious diseases, it suffers from poor compliance, potential for nosocomial infections and logistical obstacles of cost, stability, storage, distribution and disposal of used sharps. Development of needle-free, painless, safe, efficacious immunization strategies is an important goal in global health care.

Various needle-free parenteral immunization techniques have been previously described. Liquid jet injectors, such as multi-use-nozzle jet injectors (MUNJI) and disposable-cartridge jet injectors (DCJIs) use the kinetic energy of a high velocity vaccine jet (>100 m/second) to penetrate the skin and deliver the vaccine intradermally, subcutaneously or intramuscularly at a rate of up to 1,000 immunizations per hour. However serious safety issues and other disadvantages, including painful side effects and cost, have resulted in the almost abandonment of these jet injectors.

Studies have demonstrated that vaccine delivery to the skin can increase the magnitude of the immune response and in some cases, do so using less vaccine than required for intramuscular (im) injection. While traditional needles can be used to immunize via the intradermal route, this method requires extensively trained personnel and can lead to clinical variability due to controlling injection depth and administration to non-immune reactive subcutaneous tissue. Non-invasive delivery involves vaccine application to intact skin in liquid form in conjunction with a cream or patch. Of these techniques, topical vaccine administration with adjuvants, such as bacterial toxins, in the context of skin hydration has been shown to generate strong mucosal and systemic antibody responses in pre-clinical models. However, questions over the antigen doses, the need for potentially toxic adjuvants, and their long-term consequences and clinical performance have limited wide scale acceptance of this method.

Invasive particle based methods using gene guns have been developed to clinical trial. These function by delivering a powdered formulation into the epidermis. Formulation of the vaccine with dense gold beads in conjunction with cumbersome, biolistic devices limits the cost-effectiveness and utility of these delivery systems to specific groups of trained health care professionals. Other invasive skin delivery technologies that have been developed to permeabilise the stratum corneum (SC), such as iontophoresis, thermal microporation and photomechanisation require expensive or large equipment, preventing their use as mass vaccination devices or outside a research or specialised healthcare environment at this time.

There is thus a need for improved vaccination systems which overcome these disadvantages.

### Microneedles

Microneedles are solid or hollow arrays of micron scale projections ranging in height typically from 50-900 μm or more. Microneedle arrays have been fabricated from metals, silicon, silicon dioxide, biodegradable polymers as well as other materials.

While microneedles are being extensively developed for drug delivery, there has been less investigation into their use for vaccine delivery.

Widera et al ((2006) Vaccine 24, 1653-64) describe a coated microneedle array patch system. Using microneedles of 4 different designs, it was demonstrated that the induction of antibody responses was independent of depth of delivery, density of microneedles on the array or the area of application but was dependent on the dose of vaccine coated onto the delivery device. The common perception in the art, therefore, is that the design characteristics, such as the dimensions, of the microneedles have no influence on the strength or phenotype of vaccine-induced immunity.

### SUMMARY OF ASPECTS OF THE INVENTION

The present inventors have found that, contrary to the prejudice in the art, microneedle design has a profound effect on the magnitude and phenotype of cell-based immune responses induced in the vaccinated subject.

Moreover, the present inventors have shown that, by selecting particular needle characteristics and vaccination regimes it is possible to modulate the type of immune response, enabling selection of an appropriate regime, depending on the desired immunological outcome.

The present invention is based on the following findings:
Use of a microneedle array that creates a small pore volume for vaccine delivery induces a T cell response that is programmed to significantly expand during a boosting immunisation
Use of a microneedle array that creates a small pore volume for vaccine delivery induces a T cell response characterised by a higher proportion of antigen-specific T cells with a central memory phenotype (T_{CM} cells)
In an immunised individual, for example an individual previously exposed to antigen, vaccination using a large pore volume array leads to an increased immune response.

In a prime:boost vaccination regime, priming with a small pore volume array and boosting with a large pore volume array significantly enhances the T cell response compared to conventional intradermal delivery, using a needle and syringe.
In a prime:boost vaccination regime, priming with an intermediate or large pore volume array, followed by conventional intradermal boosting immunisation, significantly increases the antibody response.

The volume of the pore created by a microneedle is directly proportional to the volume of the microneedle itself. Hence, the total volume of the microneedles in the microarray device equates with the total pore volume created in the skin following application.

Thus, in a first aspect, the present invention provides a prime-boost vaccination method which comprises the following steps:
(i) administering a priming vaccine using a microneedle array having a small total microneedle volume; and
(ii) administering a boosting vaccine using a microneedle array having a large total microneedle volume.

The large total microneedle volume used to administer the boosting vaccine, may be at least two-fold, for example at least five-fold greater than the small total microneedle volume, used to administer the priming vaccine.

The small total microneedle volume may be, for example, between 0.0005 and 0.014 mm³; whereas the large total microneedle volume may be, for example, at least 0.05 mm³. The large total microneedle volume may be between 0.07 and 0.25 mm³.

The present invention also provides a kit for use in such a method, which comprises:

(i) a microneedle array suitable for administering a priming vaccine having a small total microneedle volume; and/or

(ii) a microneedle array suitable for administering a boosting vaccine having a large total microneedle volume.

The microneedle array suitable for administering a priming vaccine may, for example, have a total microneedle volume of between 0.0005 and 0.014 mm³. The microneedle array suitable for administering a boosting vaccine may, for example, have a total microneedle volume of at least 0.05 mm³.

In a second aspect, the present invention provides a prime-boost vaccination method which comprises the following steps:
(i) administering a priming vaccine; and
(ii) administering a boosting vaccine
   wherein the priming vaccine is administered using a microneedle array having a small total microneedle volume, for example a total microneedle volume of between 0.0005 and 0.014 mm³

The priming vaccine may be administered using a microneedle array which has a total needle surface area between 0.1 and 0.8 mm²

For the pyramidal wet-etch manufactured silicon microneedles described in the example, which have the approximately volume of a right circular cone, there is a relationship between maximum pore volume and surface area, as shown below:

Pore volume=0.12 h³

Pore surface area=1.02 h²

where h is the needle height.

The “pore volume” as used herein refers to the maximum volume of the conduit or indentation that can be created, through the stratum corneum, after application of a microneedle to the skin. For a single application of the array, the “pore volume” therefore equates to the total volume of the needles of the array. Where the array is applied multiple times, the pore volume is the product of the total volume of the needles of the array and the number of times the array is applied.

The boosting vaccine may be administered using a microneedle array having a large total microneedle volume, for example a total microneedle volume of at least 0.05 mm³, such as between 0.07 and 0.25 mm³.

The priming vaccine and optionally the boosting vaccine may comprise a modified vaccinia virus Ankara (MVA) vector expressing the antigen or epitope of interest.

The present invention also provides a priming vaccine kit for use in such a method, which comprises a microneedle having a small total microneedle volume, for example a microneedle volume of between 0.0005 and 0.014 mm³.

The kit may also comprise a priming vaccine composition.

The present invention also provides a prime-boost vaccine kit for use in a method according to the second aspect of the invention, which comprises:
(i) a microneedle array for administration of the priming vaccine having a small total microneedle volume, for example a microneedle volume of between 0.0005 and 0.014 mm³;
(ii) a boosting composition, and/or means for administration of a boosting composition to the subject.

The kit may also comprise a priming vaccine composition.

The kit may comprise a microneedle array for administration of the boosting vaccine having a large total microneedle volume, for example a microneedle volume of between 0.070 and 0.25 mm³.

The present invention also provides a method for inducing a T-cell response in a subject which produces an increased antigen-specific T-cell response to a boosting administration, which method comprises the step of administering a priming vaccine using a microneedle array having a small total microneedle volume, for example a total microneedle volume of between 0.0005 and 0.014 mm³.

Using this method, the T-cell response induced by the priming vaccine may have a high proportion of central memory T cells, for example an approximately equal frequency of effector memory T cells and central memory T cells.

The present invention also provides a method for inducing central memory T cells in a subject, which comprises the step of administering a priming vaccine using a microneedle array having a small total microneedle volume, for example a total microneedle volume of between 0.0005 and 0.014 mm³.

In a third aspect, the present invention provides a method for inducing an improved immune response in a subject, which comprises the step of administering a vaccine using a microneedle array having a large total microneedle volume, for example a total microneedle volume of between 0.05 and 0.25 mm³.

The subject may have been previously exposed to antigen, either naturally (for example by infection) or by way of a priming immunisation.

The present invention also provides a vaccine kit for use in such a method, which comprises a microneedle array having a large total microneedle volume, for example a total microneedle volume of between 0.05 and 0.25 mm³.

The kit may also comprise a vaccine composition, such as a boosting vaccine composition.

In a fourth aspect, the present invention provides a prime-boost vaccination method which comprises the following steps:
(i) administering a priming vaccine; and
(ii) administering a boosting vaccine
   wherein the priming vaccine is administered using a microneedle array having an intermediate or large total microneedle volume, for example a total microneedle volume of between 0.014 and 0.25 mm³.

The priming vaccine and boosting vaccine may be heterologous. For example the priming vaccine may comprise a recombinant adenovirus vector and/or the boosting vaccine may comprise a modified vaccinia virus Ankara (MVA) vector.

The boosting vaccine may be administered by an intradermal (ID) route.

The present invention also provides a priming vaccine kit for use in such a method, which comprises a microneedle array having an intermediate or large total microneedle volume, for example a total microneedle volume of between 0.014 and 0.25 mm³.

The kit may also comprise a priming vaccine composition.

The present invention also provides a prime-boost vaccine kit for use in such a method, which comprises:
(i) a microneedle array for administration of the priming vaccine having an intermediate or large total microneedle volume, for example a total microneedle volume of between 0.014 and 0.25 mm³; and
(ii) a boosting composition and/or means for administration of a boosting administration to the subject.

The kit may also comprise a priming vaccine and/or a boosting vaccine. The boosting vaccine may be for intradermal administration.

The present invention also provides a method for inducing an improved antibody response in a prime-boost vaccination method, which comprises the step of administering a priming vaccine using a microneedle array having an intermediate or large total microneedle volume, for example a total microneedle volume of between 0.02 and 0.25 mm³.

The method or kit of the present invention may be for vaccination against a range of pathogens. Such diseases include but are not limited to malaria, infection and disease caused by the viruses HIV, herpes simplex, herpes zoster, hepatitis C, hepatitis B, influenza, Epstein-Barr virus, measles, dengue and HTLV-1; by the bacteria Mycobacterium tuberculosis and Listeria sp.; by encapsulated bacteria such as streptococcus and haemophilus; and by parasites such as Leishmania, Toxoplasma and Trypanosoma; and certain forms of cancer e.g. melanoma, lymphomas and leukaemia, cancers of the lung, breast and cancer of the colon.

The present invention also provides a method for administering an adenovirus vector to a subject, which vector comprises a nucleic acid sequence encoding an antigen, the method comprising the step of using a microneedle array to administer the adenovirus vector to the subject.

The microneedle array may be used for a priming administration, for example in a homologous prime-boost regime.

The present inventors have found that priming with adenovirus vector using a microneedle array induced a significantly decreased antibody response to adenovirus vector compared to intradermal administration.

The method of the present invention may give an increased ratio of anti-antigen to anti-vector antibody response as compared to an equivalent immunisation program using intradermal immunization. The ratio may be increased by at least 5, 10, 50 or 100-fold. The ratio may be measured by any suitable technique (such as ELISA) at an appropriate time point (such as 50 days) following immunisation.

The present invention also provides a method for increasing the ratio of anti-antigen to anti-vector antibody response obtained following immunization with an an adenovirus vector to a subject, which vector comprises a nucleic acid sequence encoding an antigen, wherein the ratio is increased as compared to an equivalent immunisation program using intradermal immunization of the vector, which method comprises the step of using a microneedle array to administer the adenovirus vector to the subject.

The present invention also provides a kit for use in method according to the invention, which comprises (i) a microneedle array; and (ii) an adenovirus vector, which comprises a nucleic acid sequence encoding an antigen.

The adenovirus vector may be coated on to the exterior surface of the microneedles. Alternatively the kit may comprise a composition comprising the adenovirus vector for application to the skin before or after application of the microneedle array.

### DESCRIPTION OF THE FIGURES

FIG. 1A: CD8⁺IFN⁺ and CD8⁺TNF-α⁺ as a % of CD8⁺ two weeks post-prime

FIG. 1B Memory Phenotype post-prime

FIG. 1C: Ratio of the frequency of T_{EM} to T_{CM}

FIG. 2: Post-boost CD8⁺IFN⁺ and CD8⁺TNF-α⁺ as a % of CD8⁺

FIG. 3: CD8⁺ cytokine+ T cells as % CD8⁺

FIG. 4A: pre-boost Ab titres Ad-MSP

FIG. 4B: Post-boost titres; Ad/MVA-MSP#

FIG. 4C: Anti-vector antibody titres, 50 days post-prime

FIG. 4D: Ratio of anti-antigen to anti-vector antibody titres, 50 days post-prime

FIG. 5: Optimum order of vaccine and array application

FIG. 6: Differential distribution of fluorescence detected in pig skin when MVA expressing red fluorescent protein (MVA-RFP) is delivered by microneedle array compared to id

FIG. 7: Delivery dose of microneedle array.

### DETAILED DESCRIPTION

### Percutaneous Delivery

The function of the skin is to protect against water loss and act as the first line of defence against the entry of pathogens into the body. Mammalian skin can be subdivided into three layers; the stratum corneum (SC); in humans this is 10-20 μm in depth, the viable epidermis (50-100 μm in humans) and the dermis (1-3 mm in humans). The stratum corneum is composed of closely packed dead keratinocytes embedded in a highly organized intercellular lipid matrix that forms a barrier that is impermeable to microbes and large molecules such as vaccine antigens. In order to exert an immune response it is necessary to get the vaccine across the stratum corneum (SC).

A rich network of innate immune cells, such as Langerhans cells (LCs), monocytes and dermal dendritic cells (DC), reside in the underlying epidermis and dermis. Intradermal vaccination with needle and syringe (ID) can induce quantitatively or qualitatively superior immunity compared to intramuscular (IM) or subcutaneous (SC) delivery; this has been exemplified in particular for antibody-inducing influenza vaccines.

The present invention relates to percutaneous delivery of a vaccine. Percutaneous administration is through the skin and, as such, is distinct from topical administration, which involves administration to the outer surface of the skin. Transdermal administration involves administration though the dermal layer of the skin to the systemic circulation by diffusion. The methods of the present invention, on the other hand do not specifically aim to target the vaccine to go to the systemic circulation.

### Microneedles

A microneedle array is a device for delivering an agent through the stratum corneum of the skin, which comprises a substantially flat base plate, on which is mounted a plurality of microprotrusions. Upon application to the skin, the microprotrusions form a plurality of micropathways or microconduits extending through the stratum corneum, and either into the epidermis, or through the hypodermis into the underlying dermis.

The vaccination kits/methods of the present invention may use any appropriate form of microneedle array, such as solid silicon microneedles fabricated by wet-etch methods, as described in US2007/0134829A1 (Wilke and Morrissey).

The microneedles used in the present invention puncture as opposed to abrade the skin. The term “abrasion” as used herein refers to disruption of the outer layers of the skin, for example by scraping or rubbing, resulting in an area of disrupted stratum corneum. Puncturing, on the other hand, produces discrete holes through the stratum corneum, with areas of undisrupted stratum corneum between the holes.

Abrasion of the skin using, for example, microelectromechanical (MEMS) devices as described in WO 01/89622 is likely to induce an inflammatory response in the skin. The method of the invention, which involves puncturing discrete holes in the skin, does not induce a significant inflammatory response at the administration site.

The microneedle device of the present invention may be applied to the skin once for a given vaccination step, such as immunising, priming or boosting. Abrasion techniques, on the other hand, often involve multiple applications of a device (for example, six to twelve times) maybe in multiple orientations, in order to strip away the stratum corneum.

The microneedle array may be applied to the skin in a single rolling motion, or by simply pushing the array substantially vertically on to the skin, as described in Haq et al ((2009) Biomed Microdevices 11:35-47). By contrast, abrasion techniques may involve lateral or rotational movement of the array to disrupt the stratum corneum (see, for example, US 2004/0077994)

In the methods of the invention, the vaccine composition may be applied to the skin before or after the microneedle array has been applied and removed from the skin. As shown in Example 5, applying the vaccine to the skin prior to application of the array can induce a stronger immune response compared to administering a vaccine subsequent to creating the temporary channels using a microneedle array.

The microneedle array may be applied to the skin of the subject in its normal state. It should not be necessary to stretch or apply tension to the subject's skin during penetration of the microneedles.

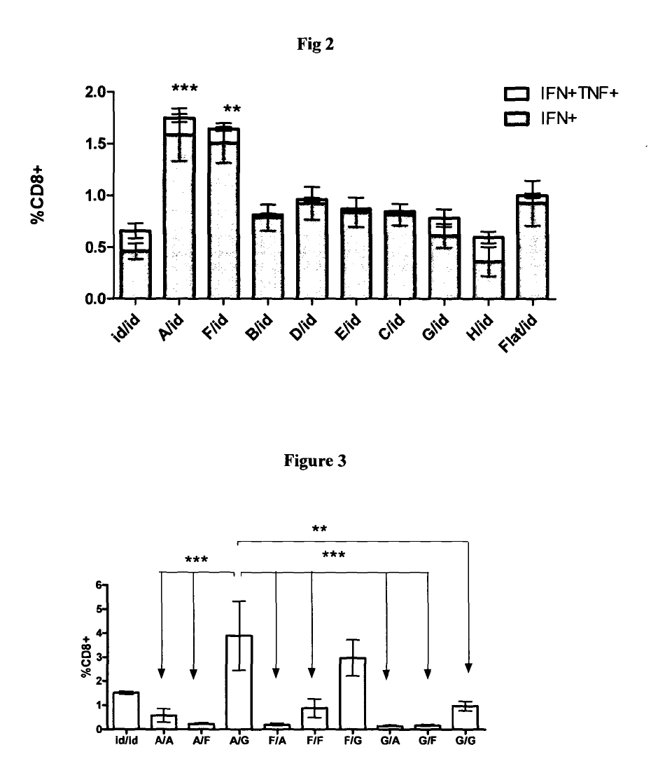

The microneedle array may be applied by hand, for example, by applying gentle pressure with a forefinger, or an applicator may be used such as those described in Haq et al (2009, as above, FIG. 2). It should not be necessary to apply the microneedle array with impact, for example using a device such as the one described in WO 02/30301.

The microneedle array may be part of a “patch”, for example a band-aid style patch, for application to the skin of a subject.

### Microneedle Dimensions and Array Characteristics

The microneedle array used in the methods of the invention may have a total area of between 10 and 100 mm², for example 20-60 mm². The array may be 3-10 mm×3-10 mm in size, for example 5.4 mm×5.4 mm or 7.4×7.4 mm.

The microprojections may be any shape which is suitable for piercing the skin and forming a microprotrusion, without significant shearing or breaking in use (unless it is biocompatible). The microprojections may be tapered, coming to a point at one end for skin piercing. The point may be sharp or blunt (a frustum). The microprojections may, for example, be substantially conical or pyramidal in shape.

The height of the microneedles may be in the range from 50 μm to 900 μm, for example 50-400 μm, 100-300 μm or 200-300 p.m.

The number of microneedles per array may range from 10-200, for example 16-100 per array.

The density of the microneedles on the array may be between 80-1000 needles per cm², for example 80-200 needles per cm².

Each microprojection may have a surface area of between 0.005 and 0.5 mm², for example between 0.01 and 0.1 mm², such that the total surface area of the combination of microprojections on the device is between the range of 0.1 mm² and 7.5 mm², for example between 0.1 and 7 mm².

Each microprojection may have a volume of between 0.0001 mm³ and 0.01 mm³, for example between 0.0001 and 0.003 mm³ such that total pore volume created in the skin by insertion of the entire microprojection array is between the range of 0.0005 and 0.25 mm³ (see below)

### Pore Volume/Total Microneedle Volume

The present inventors have found that the total pore volume (PV) created by a microneedle array has a significant impact on the magnitude and type of immune response generated in the subject.

A microneedle array that creates a “small” PV for vaccine delivery induces a T cell response that is programmed to significantly expand during a boosting immunisation. The “small” PV immune response is characterised by a higher proportion of antigen-specific T cells with a central memory phenotype (T_{CM} cells).

In a prime:boost vaccination regime, priming with a “small” PV array and boosting with a “large” PV array significantly enhances the T cell response compared to conventional intradermal delivery.

In an immunised individual, for example an individual previously exposed to antigen, vaccination using a “large” PV array leads to an increased immune response.

In a prime:boost vaccination regime, priming with an “intermediate” or “large” PV array, followed by conventional intradermal boosting immunisation, significantly increases the antibody response.

The total PV termed “small”, “intermediate” and “large” may be defined with reference to an internal standard. For example, in a prime boost regime which involves priming with an array which creates a small PV and boosting with an array which creates a large PV, the pore volume created by the large PV array is greater than that created by the small PV array. The volume created by the large PV array may be at least 2-fold, 5-fold or 10-fold greater than that created by the small PV array. The volume created by the large PV array may be between 10-100× greater than that created by the small PV array.

The volume created by an intermediate PV array may be 2-30× greater, for example 10-20× greater than the volume created by a small PV array. The volume created by a large PV array may be 2-10×, for example 5-8× greater than the volume created by an intermediate PV array.

As discussed above, the total pore volume created by an array equates to the total microneedle volume of the array, i.e. the sum of the volume of each microneedle in the array.

The volume of a microneedle may be calculated by simple mathematical mens, depending on the shape of the microneedles. For example, for conical microneedles, the volume of each microneedle will be:

1/3πr²h

where r is the radius of the cone at the bottom of the base, and h is the height of the microneedle from base to tip.

Thus, in a prime boost regime which involves priming with an array which creates a small PV and boosting with an array which creates a large PV, the large PV array has a greater total microneedle volume than that of the small PV array. The total microneedle volume of the large PV array may be at least 2-fold, 5-fold or 10-fold greater than that of the small PV array. The total microneedle volume of the large PV array may be between 10-100× greater than that of the small PV array.

The total microneedle volume of an intermediate PV array may be 2-30× greater, for example 10-20× greater than the volume of a small PV array. The total microneedle volume of a large PV array may be 2-10×, for example 5-8× greater than the volume of an intermediate PV array.

The ranges defined as “small”, “intermediate” or “large” in terms of pore volume, may be defined experimentally by testing a series of pore volumes and observing the magnitude and phenotype of the immune response generated. From the characteristics listed above, it should then be possible to assign certain PV ranges as “small”, “intermediate” or “large”.

Devices tested in animal studies have been successfully used in humans (Widera et al (2100) 28:159-165) suggesting that the microneedle design described herein should translate across species. However, if the microneedle volume requires scaling up for use in a different target tissue, the volume range for each array type i.e., small, intermediate or large; may be, for example, 2, 4, or 10-fold greater than that described here.

In connection with the present invention, an array having a “small” total microneedle volume may have a total microneedle volume of between 0.0005 and 0.014 mm³, for example between 0.001 and 0.014 mm³.

In connection with the present invention, an array having an “intermediate” total microneedle volume may have a total microneedle volume of between 0.014 and 0.05 mm³.

In connection with the present invention, an array having a “large” total microneedle volume may have a total microneedle volume of more than 0.05 mm³, for example between 0.05 and 0.5 mm³, such as between 0.07 and 0.25 mm³.

### Vaccine Composition

The vaccine composition may comprise a whole organism vaccine, comprising a live, killed or attenuated pathogen.

The vaccine composition may comprise a subunit of a pathogen, or a peptide or polypeptide derivable therefrom comprising one or more antigenic epitope(s). The vaccine composition may comprise a nucleotide sequence, such as an RNA or DNA sequence capable of encoding a peptide or polypeptide comprising one or more antigenic epitope(s).

The vaccine composition may comprise a vector capable of delivering such a nucleotide sequence to a target cell, such as a plasmid, a viral vector, a bacterial vector or a yeast vector.

The vaccine composition may comprise one or more viral vectors.

Viral vectors or viral delivery systems include, for example, adenoviral vectors, adeno-associated viral (AAV) vectors, herpes viral vectors, retroviral vectors (including lentiviral vectors) baculoviral vectors and poxvirus vectors.

The priming and boosting compositions may be identical in that they may both contain the priming source of antigen and the boosting source of antigen as defined above. A single formulation which can be used as a primer and as a booster will simplify administration.

In EP-A-0979284, it was shown that the greatest immunogenicity and protective efficacy is surprisingly observed with non-replicating vectors, rather than replicating vectors that had been used previously. Non-replicating vectors have an added advantage for vaccination in that they are in general safer for use in humans than replicating vectors.

The priming and boosting compositions described may advantageously comprise an adjuvant. In particular, a priming composition comprising a DNA plasmid vector may also comprise granulocyte macrophage-colony stimulating factor (GM-CSF), or a plasmid encoding it or other cytokines, chemokines or growth factors, to act as an adjuvant; beneficial effects are seen using GM-CSF in polypeptide form.

Alternative suitable non-viral vectors for use in the priming composition include lipid-tailed peptides known as lipopeptides, peptides fused to carrier proteins such as KLH either as fusion proteins or by chemical linkage, antigens modified with a targeting tag, for example C3d or C4b binding protein, whole antigens with adjuvant, and other similar systems. Adjuvants such as QS21 or SBAS2, also known as AS02 or AS02A. (Stoute et al. 1997 N Engl J Medicine 226: 86-91) or alum may be used with proteins, peptides or nucleic acids to enhance the induction of T cell responses. These systems are sometimes referred to as “immunogens” rather than “vectors”, but they are vectors herein in the sense that they carry relevant CD8+ T cell epitopes.

When used in the context of prime:boost protocols, the methods of the invention may utilise either homologous or heterologous prime boost immunization regimes.

The term “vectored vaccines” is well known in the art and includes plasmid DNA, recombinants of poxviruses such as MVA, replicating vaccinia, fowlpox, avipox, also of adenoviruses including non-human primate adenoviruses, of alphaviruses, of vesicular stomatitis virus, and bacterial vectors such as Salmonella, Shigella and BCG.

Examples of viral vectors that are useful in this context are vaccinia virus vectors such as MVA or NYVAC. A preferred viral vector is the vaccinia strain modified virus ankara (MVA) or a strain derived from MVA. Alternatives to vaccinia vectors include avipox vectors such as fowlpox or canarypox vectors.

Preferably, the vector used in the method according to the invention is a non-viral vector or a non-replicating or replication-impaired viral vector. In the case of prime boost protocols, the source of antigen in the priming composition is preferably not the same poxvirus vector or not a poxvirus, so that there is minimal cross-reactivity between the primer and the booster. Further details of preferred protocols for use with prime boost vaccines are disclosed in EP-A-0979284.

The term “non-replicating” or “replication-impaired” as used herein means not capable of replication to any significant extent in the majority of normal mammalian cells or normal human cells. Viruses which are non-replicating or replication-impaired may have become so naturally (i.e. they may be isolated as such from nature) or artificially e.g. by breeding in vitro or by genetic manipulation, for example deletion of a gene which is critical for replication. There will generally be one or a few cell types in which the viruses can be grown, such as CEF cells for MVA.

### Poxvirus Vectors

The vaccine may comprise a recombinant poxvirus vector. The vaccine may comprise a non-replicating or replication impaired viral vector such as MVA.

Examples of poxviruses include MVA, NYVAC, avipox viruses and the attenuated vaccinia strain M7.

Modified vaccinia virus Ankara (MVA) is a strain of vaccinia virus which does not replicate in most cell types, including normal human tissues. MVA was derived by serial passage over 500 times in chick embryo fibroblasts (CEF) of material derived from a pox lesion on a horse in Ankara, Turkey (Mayr et al. Infection (1975) 33: 6-14.). It was shown to be replication-impaired yet able to induce protective immunity against veterinary poxvirus infections. MVA was used as a human vaccine in the final stages of the smallpox eradication campaign, being administered by intracutaneous, subcutaneous and intramuscular routes to more than 120,000 subjects in southern Germany. No significant side effects were recorded, despite the deliberate targeting of vaccination to high risk groups such as those with eczema (Mayr et al. Bakteriol B. (1978)167: 375-90).

Another replication-impaired and safe strain of vaccinia known as NYVAC is fully described in Tartaglia et al. (Virology 1992, 188: 217-232).

Poxvirus genomes can carry a large amount of heterologous genetic information. Other requirements for viral vectors for use in vaccines include good immunogenicity and safety. In one embodiment the poxvirus vector may be a fowlpox vector, or derivative thereof.

It will be evident that vaccinia virus strains derived from MVA, or independently developed strains having the features of MVA which make MVA particularly suitable for use in a vaccine, will also be suitable for use in the invention.

MVA containing an inserted string of epitopes has been previously described in WO 98/56919.

Alternative preferred viral vectors for use in the priming composition according to the invention include a variety of different viruses, genetically disabled so as to be non-replicating or replication impaired. Such viruses include for example non-replicating adenoviruses such as El deletion mutants. Genetic disabling of viruses to produce non-replicating or replication-impaired vectors has been widely described in the literature (e.g. McLean et al (1994) J Infect Dis. 170(5):1100-9).

Other suitable viral vectors for use in the priming composition are vectors based on herpes virus and Venezuelan equine encephalitis virus (VEE). Suitable bacterial vectors for priming include recombinant BCG and recombinant Salmonella and Salmonella transformed with plasmid DNA (Darji A et al. 1997 Cell 91: 765-775).

### Adenovirus Vectors

Adenoviruses form the family Adenoviridae. All adenoviruses have a similar virion—medium-sized (60-90 nm), non-enveloped, icosahedral particles, with a protein capsid (240 hexons and 12 pentons) enclosing a 34-43 kbp double-stranded DNA genome within the core (Tatsis, and Ertl. 2004. MoI Ther 10:616). Fifty-one human adenovirus (AdHu) serotypes have so far been described, based on serological studies of cross-neutralising antibody responses to the hexon protein and terminal knob of the penton fibre. These serotypes have been further grouped into six subgroups or species (A-F). The adenoviral genome is well characterised and comparatively easy to manipulate (Ghosh-Choudhury et al., 1986. Gene 50:161; Graham, and Prevec. 1995. MoI Biotechnol 3:207). Deletion of crucial regions of the viral genome, such as El, renders the vectors replication-defective, which increases their predictability and eliminates unwanted pathogenic side effects. The adenoviral capsid will only allow a 5% increase in genome size before efficient packaging and viral stability is disrupted—an extra 1.8 kbp in the case of the well-studied vector AdHu5 (Tatsis and Ertl. 2004. MoI Ther 10:616). Vectors deleted of El and the nonessential E3 region (Saito et al., 1985. J Virol 54:711.21) can accommodate up to 7.5 kbp of foreign DNA and remain the leading choice for vaccine studies using this vector. Replication-deficient adenoviruses can be grown to high titre in tissue culture, using cell lines that provide the missing essential El gene products in trans (Fallaux et al., 1998. Hum Gene Ther 9:1909). They can be applied systemically as well as through mucosal surfaces and their relative thermostability facilitates their clinical use. The majority of studies now focus on the most promising adenovirus vectors of differing human serotype, including AdHu5 and AdHu35 that can induce potent and protective T and B cell-mediated responses against a range of viral and parasitic encoded antigens (Draper et al (2009) Cell Host Microbe 5, 95-105 and (2008) Nat Med 14, 819-21; Ophorst et al., 2006. Infect Immun 74:313. 15; Rodrigues et al., 1997. J Immunol 158:1268; Shiver et al., 2002, Nature 415:331).

### Prime-Boost Vaccination Regimes

One of the most important properties of adaptive immunity is immunological memory, the ability to respond more rapidly and more intensely on a second encounter with a pathogen.

This phenomenon is utilised in vaccination strategies, where it is common to give a first “priming” immunisation to a subject, which primes the immune system for subsequent challenge, followed by a second “boosting” immunisation, which induces a faster, greater and more immunologically focussed immune response.

In a homologous prime-boost vaccination regime, the same vaccine is administrated to the subject in both the priming and boosting immunisations.

In a heterologous prime-boost vaccination regime, the vaccine given in the priming immunisation is different from the vaccine given in the boosting immunisation, but may be based on the same pathogen, antigen or antigenic epitope. For example, the priming immunisation may involve a live-vector vaccine, and the boosting immunisation may involve a recombinant subunit vaccine. Alternatively, the priming immunisation may involve DNA vaccination, and the boosting immunisation may be via a recombinant viral vector. The DNA vaccine and recombinant viral vector may cause expression of the same antigen or antigenic epitope(s).

The present inventors have previously developed heterologous prime-boost vaccine strategies against a range of pathogens including Plasmodium spp. that causes malaria, Mycobacterium tuberculosis and Hepatitis B Virus. These vaccine strategies are based on the sequential use of different vaccines including the same antigen or epitope of interest such as the use of recombinant protein antigen or plasmid DNA or non-replicating virus vectors that express the antigen of interest.

With respect to malaria vaccines, they have found that a priming vaccination with recombinant DNA or adenoviruses followed by a boosting immunization with recombinant Modified Vaccinia virus Ankara (MVA) is highly efficacious, inducing sterile immunity in a mouse model and in some humans.

The term “vaccine” encompasses both a prophylactic composition for the prevention of a disease and a therapeutic composition for the treatment of an existing disease.

To “treat” means to administer the vaccine to a subject having an existing disease in order to lessen, reduce or improve at least one symptom associated with the disease and/or to slow down, reduce or block the progression of the disease.

To “prevent” means to administer the vaccine to a subject who has not yet contracted the disease and/or who is not showing any symptoms of the disease to prevent or impair the cause of the disease (e.g. infection) or to reduce or prevent development of at least one symptom associated with the disease.

### T-Cell Responses

CD4⁺ T helper cells (T_{H} cells) assist other white blood cells in immunological processes, including maturation of B cells into plasma cells and activation of cytotoxic T cells and macrophages. T_{H} cell recognize antigen presented in conjunction with MHC class II molecules by antigen presenting cells. Once activated, T_{H} cells divide rapidly and secrete cytokines that regulate or assist in the immune response. These cells can differentiate into one of several subtypes, including T_{H}1, T_{H}2, T_{H}3, T_{H}17 and T_{FH}, which secrete different cytokines to facilitate a different type of immune response.

Protection against pathogen challenge in the case of diseases such as malaria, tuberculosis and hepatitis correlates with the induction of a T cell responses characterised by the production of high levels of IFN-γ. More recently, vaccines that induce multi-functional CD4⁺ T cell responses, where antigen-specific T cells produce more than one effector cytokine; IFN-γ, IL-2 and TNF-α, were demonstrated to be more efficacious than vaccines that induced T cells that only produced a single cytokine (Darrah et al (2007) Nat Med 13: 843-50).

Cytotoxic T cells (T_{C} cells, or CTLs), which are CD8⁺, are involved in the clearance of virally infected cells and tumor cells, and are also implicated in transplant rejection. T_{C} cells recognize their targets by binding to antigen associated with MHC class I, which is present on the surface of nearly every cell of the body.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells can be described as different subtypes: central memory T cells (T_{CM} cells) and effector memory T cells (T_{EM} cells). Memory T cells may be either CD4⁺ or CD8⁺. T cell subsets may be defined according to their expression of various markers, for example CD62L and CD127 expression may be used to distinguish: central memory T cells (T_{CM}: CD62L⁺ CD127⁺), that reside in lymphoid tissue; effector memory T cells (T_{EM}: CD162L⁻ CD127⁺), that circulate into peripheral, non-lymphoid tissue as well as lymphoid tissues; and effector T cells (T_{E}: CD62L⁻ CD127⁻)(Bachman et al., J. Immunology, 2005, 175:4686-4696).

T cell subsets can also be distinguished according to their cytokine profile. For example cytokines associated with CD8+ T cells include IFNγ, TNFα and IL-2. T cells that produce IL-2 are believed to be from the memory subset.

In connection with the present invention:

i) immunisation with a large PV (i.e. an array having a large total microneedle volume) results in a higher frequency of CD8+IFNγ+ T cells, and CD8+ cells which produce one or more of IFNγ, TNFα and IL-2, within the total CD8+ T cell population after a single immunisation;

ii) immunisation with a small PV (i.e. an array having a small total microneedle volume) results in a higher frequency of antigen-specific T cells with the central memory phenotype (CD62L⁺CD127⁺CD8⁺ T cells);

iii) immunisation with a large PV (i.e. an array having a large total microneedle volume) induces a predominantly T_{EM} response, which has a higher frequency of T_{EM} cells compared to T_{CM} cells, for example at least 2-fold or 5-fold higher frequency of T_{EM} cells compared to T_{CM} cells

iv) immunisation with microneedle arrays that create small PVs (i.e. an array having a small total microneedle volume) induces an immune response that a relatively high frequency of T_{CM} CD8⁺ T cells, for example, it may have an approximately equal frequency of T_{EM} and T_{CM} CD8⁺ T cells;

v) priming with an array that creates a small PV (i.e. an array having a small total microneedle volume), followed by intradermal boosting, gives a post-boost immune response characterised by a high frequency of CD8⁺IFN-γ⁺ T cells and a high frequency of CD8⁺cytokine⁺ T cells (CD8+ T cells expressing IFN-γ TNF-α, and/or IL-2);

vi) priming with an array that creates a small PV (i.e. an array having a small total microneedle volume), followed by boosting with an array that creates a large PV (i.e. an array having a large total microneedle volume) induces significantly greater T cells responses than intradermal delivery or any other prime-boost microneedle array combination.

### Antibody Responses

When a naïve B cell encounters antigen and an additional signal from a T helper cell, it can further differentiate into either plasma B cells or memory B cells. A single B cell or a clone of cells with shared specificity upon encountering its specific antigen may divide to produce many B cells. Most of such B cells differentiate into plasma cells that secrete antibodies into blood that bind the same epitope that elicited proliferation in the first place. A small minority survives as memory cells that can recognize only the same epitope. However, with each cycle, the number of surviving memory cells increases. The increase is accompanied by affinity maturation which induces the survival of B cells that bind to the particular antigen with high affinity. This subsequent amplification with improved specificity of immune response, which is known as the secondary immune response, may be triggered deliberately by using a prime-boost vaccination regime.

The quantity and quality of the antibody response may be determined by methods known in the art.

An antibody titer is a measurement of how much antibody an organism has produced that recognizes a particular epitope, expressed as the greatest dilution ratio (or its reciprocal) that still gives a positive result. ELISA is a common means of determining antibody titers

In the context of the present invention, vaccination with an array that creates an intermediate pore volume (i.e. an array having an intermediate total microneedle volume) resulted in increased antibody titres. In a prime-boost vaccination regime, priming with intermediate or large PV arrays, followed by id boosting, gave a significant increase in post-boost antibody titre.

### Kits

The present invention also provides kits for use in the methods of the present invention.

In a first embodiment, the present invention provides a priming vaccine kit, which comprises a microneedle array having small total microneedle volume. For example, it may have atotal microneedle volume of between 0.0005 and 0.014 mm³, such as between 0.0018 and 0.0113 mm³.

The kit may also comprise a priming vaccine composition.

In a second embodiment the present invention provides a prime-boost vaccine kit, which comprises:
(i) a microneedle array according to the first embodiment; and
(ii) means for administrating a boosting composition to the subject.

The kit may also comprise the priming and/or boosting vaccine compositions.

The means for administrating a boosting composition may comprise a microneedle array having a large total microneedle volume. For example, it may have a total microneedle volume of between 0.07 and 0.25 mm³, such as between 0.070 and 0.205 mm³.

In a third embodiment the present invention provides a vaccine kit which comprises a microneedle array having a total microneedle volume of between 0.07 and 0.25 mm³, for example between 0.07 and 0.205 mm³.

In a fourth embodiment the present invention provides vaccine kit for use in a method to induce improved antibody responses, which comprises a microneedle array having an intermediate or large total microneedle volume, for example a total microneedle volume of between 0.014 and 0.25 mm³.

The array may be used for administration of a priming immunisation. The kit may also comprise means for administrating a boosting immunisation, for example via the intradermal route.

The kit may also comprise a vaccine composition for single administration, or a priming and/or boosting vaccine composition.

Kits of the present invention may also comprise instructions for use.

### Subject

The subject may be a mammalian subject, in particular a human, or a domestic or livestock animal such as a cat, dog, rabbit, guinea pig, rodent, horse, goat, sheep, cow or pig, or wild species, such as badgers. For veterinary applications, the array may be applied to an area on the animal which has little hair, such as the inner ear or the footpad, or it may be necessary to remove hair from the skin prior to patent application.

The subject may be a human subject, in particular suffering from or at risk from contracting a particular disease. The subject may be a child or an adult subject.

The subject may be a healthy subject, believed to be at risk from contracting a disease. Alternatively the subject may already have or have had a disease.

The subject may have been previously exposed to antigen either by contact with the pathogen (for example by infection) or by prior immunisation.

### Diseases, Pathogens and Antigens

The target antigen may be characteristic of the target disease. If the disease is an infectious disease, caused by an infectious pathogen, then the target antigen may be derivable from the infectious pathogen.

The target antigen may be an antigen which is recognised by the immune system after infection with the disease. Alternatively the antigen may be normally “invisible” to the immune system such that the method induces a non-physiological T cell response. This may be helpful in diseases where the immune response triggered by the disease is not effective (for example does not succeed in clearing the infection) since it may open up another line of attack.

The antigen may be derivable from M. tuberculosis. For example, the antigen may be Antigen 85A, ESAT6 or MPT63. Alternatively, the antigen may be derivable from the malaria-associated pathogens Plasmodium berghei (e.g. the circumsporozoite protein (PbCSP)), P. Falciparum; or from the Hepatitis B virus.

The compositions of the present invention may comprise T cell or B cell epitopes from more than one antigen. For example, the composition may comprise one or more T or B cell epitopes from two or more antigens associated with the same disease. The two or more antigens may be derivable from the same pathogenic organism.

The composition may, on the other hand, comprise epitopes derivable from different pathogens, for inducing immune responses against a plurality of diseases.

The invention will now be further described by way of Examples, which are meant to serve to assist one of ordinary skill in the art in carrying out the invention and are not intended in any way to limit the scope of the invention.

### EXAMPLES

### Example 1

### Microneedle Arrays with Smaller Total Microneedle Volumes Induce a Stronger Memory T Cell Response after a Single Immunisation

Female BALB/c mice were immunized with a poxvirus, MVA, expressing a dominant MHC class I epitope (termed Pb9) from Plasmodium berghei circumsporozoite protein (PbCSP) either by needle and syringe via the intradermal (id) route or by delivering it through the stratum corneum (SC) using a microneedle array with the dimensions described in Table 1. CD8⁺ T cell responses to the dominant Pb9 epitope were assessed in spleens of immunized mice two weeks post-immunization by flow cytometry analysis (FACS). Spleen cells were restimulated with antigen and subsequently surface stained with antibodies to CD8, CD62L, CD127 and stained intracellularly for IFN-γ, TNF- [symbol] and IL-2. In this manner, the magnitude of cytokine responses can be assessed and the memory phenotype of antigen-specific T cells analysed. Antigen-specific, cytokine expressing central memory CD8⁺ T cells (T_{CM}) are CD62L^{hi}CD127⁺, effector memory T cells (T_{EM}) cells are CD62L⁻CD127^{hi} and effector T cells (T_{E}) are CD62L⁻CD127⁻

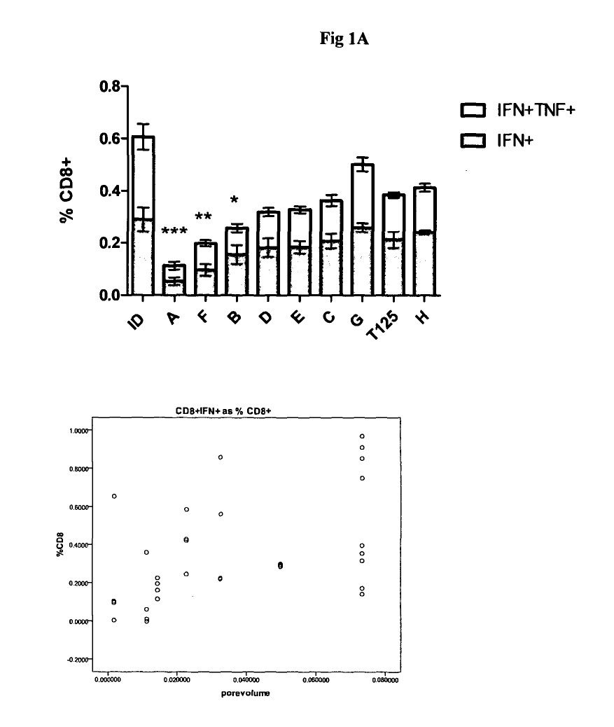

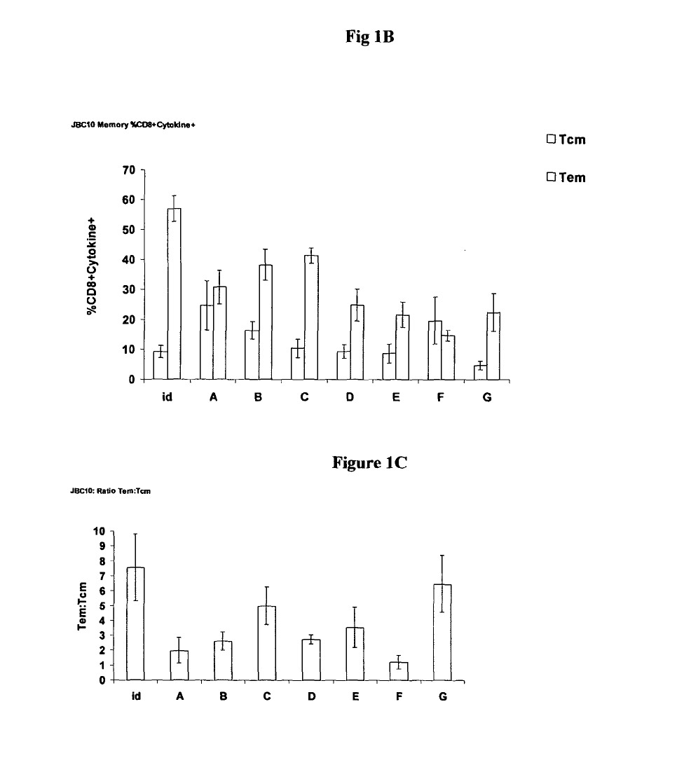

After a priming immunization of a naïve host, the frequency of CD8⁺ T cells that produce antigen-specific IFN-γ significantly correlates with the total pore volume of the microneedle array. (Spearman's rho=0.675, p<0.0005) (FIG. 1A). Thus a larger pore volume, for example as in the Array G design, results in a higher frequency of CD8⁺IFN-γ⁺T cells within the total CD8⁺ T cell population after a single immunization. In addition, the frequency of CD8⁺ T cells that produce IFN-γ TNF-α, and IL-2 alone or as a combination of two or three cytokines in response to antigen stimulation, termed “CD8⁺cytokine⁺” also significantly correlates with the total pore volume of the microneedle array. (Spearman's rho=0.591, p<0.0005) (FIG. 1B).

It is now recognised within the field that specific subpopulations of T cells respond more vigorously to secondary antigen stimulation. Specifically, it is believed that central memory T cells, “T_{CM}”, that express high levels of CD62L and CD127 on their cell surface (CD62L⁺CD127⁺CD8⁺ T cells) demonstrate significant re-expansion on antigen re-stimulation (Badovinac et al (2005) Nat Med 11, 748-56). Hence, the memory phenotype of antigen-specific T cells that were induced by id or microneedle array vaccination was investigated. In contrast to id immunization, the use of microneedle arrays that created small pore volumes (exemplified by array A and F) induced a higher frequency of antigen-specific T cells with a T_{CM} phenotype. Vaccination with microneedle arrays that created intermediate pore volumes (e.g., arrays B, C, D, E) induced a frequency of T_{CM} cells similar to id immunization, whereas the use of a microneedle array with large pore volume (e.g. Array G) resulted in a decreased T_{CM} response after a priming immunization. Therefore, while immunization with microneedle arrays that create small pore volumes induces an immune response that has a lower frequency of cytokine-producing T cells, within this population of antigen-specific cells a greater percentage of these cells possess a central memory phenotype (T_{CM}).

Intradermal immunization with MVA-PbCSP induces a predominantly T_{EM} response, with an approximate 6-fold higher frequency of T_{EM} cells compared to T_{CM} cells (FIG. 1C). Immunization using a microneedle array that creates a large pore volume (array G) also induced this relative frequency of T_{EM} to T_{CM} of approximately 6 times more T_{EM} compared to T_{CM} (FIG. 1D). However, immunization with microneedle arrays that create small pore volumes induces an immune response that has an approximate equal frequency of T_{EM} and T_{CM} CD8⁺ T cells.

It was then tested which microneedle array induced a primary immune response that responded more favourably to a boosting immunization. Mice were primed with MVA-PbCSP by the id route or with a specific microneedle array and then boosted by the id route with the same vaccine 2 weeks after the priming vaccination. It was discovered that immunization with microneedle arrays possessing a small pore volume (exemplified by array A and array F) induced a significantly increased T cell response, as assessed by the frequency of antigen-specific CD8⁺IFN-γ⁺ and CD8⁺TNF-α⁺ T cells (FIG. 2). Vaccine administration using intermediate or large pore volume microneedle arrays resulted in an equivalent immune response to id delivery. A negative correlation was observed between total pore volume and the post-boost frequency of CD8⁺IFN-γ⁺T cells (Spearman's r=−0.358, p=0.013) and also with the frequency of CD8⁺cytokine⁺ T cells (Spearman's r=−0.384, p=0.008). Thus delivering a vaccine with a microneedle array designed to have a smaller pore volume will induce a stronger post-boost T cell response. As a control, vaccine was delivered percutaneously using a silicon array (5.4×5.4 mm in area) that had no microneedle projections etched onto it, termed ‘flat’. The induction of antigen-specific T cells was significantly abrogated, compared to id immunization by this method.

### Example 2

### Array Prime/Array Boost

With a view to developing an “all-array” vaccination protocol, to eliminate the use of hypodermic needles, the best combination of arrays was investigated to use for repeated immunizations. Female BALB/c mice were immunized with a poxvirus expressing a dominant MHC class I epitope (termed Pb9) from Plasmodium berghei circumsporozoite protein (PbCSP) either by needle and syringe via the id route or by delivering it through the SC using a microneedle array with the dimensions described in Table 1. Animals were boosted after 14 days with a microneedle array with a total pore volume of 3.54 mm³ (array G). CD8⁺ T cell responses to the dominant Pb9 epitope were assessed in spleens of immunized mice two weeks post-immunization by flow cytometry analysis (FACS). Spleen cells were restimulated with antigen and subsequently surface stained with antibodies to CD8, CD62L, CD127 and stained intracellularly for IFN-γ, TNF- [symbol] and IL-2. The frequency of all cytokine expressing CD8⁺ T cells is increased when vaccine was delivered using a microneedle array total pore volume of 0.0018 mm³ or 0.0113 mm³ and boosted with a microneedle array with a total pore volume of 3.54 mm³. Hence, priming with a small pore volume array and boosting with large pore volume array approximately doubles the immune response compared to id delivery (FIG. 3). Priming with a small pore volume and boosting with a large pore volume microneedle array can induce significantly greater T cell responses compared to any other prime-boost microneedle array combination. The use of a small pore volume array in the prime and in the boost induces a weaker post-boost immune response compared to id. In contrast, vaccination using a large volume array of mice primed with a small or large pore volume array resulted in the induction of greater or equivalent responses to 2 id immunizations.

### Example 3

### Antibody Responses

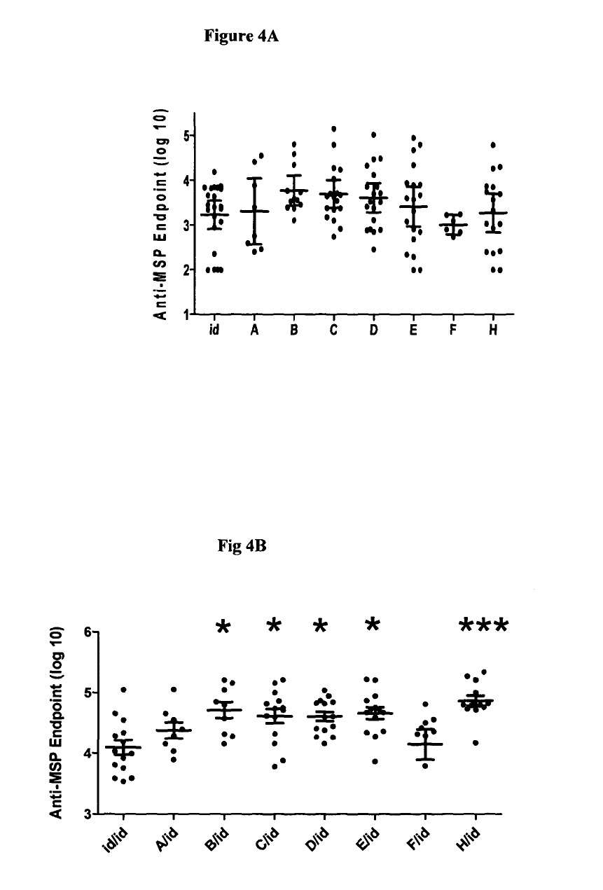

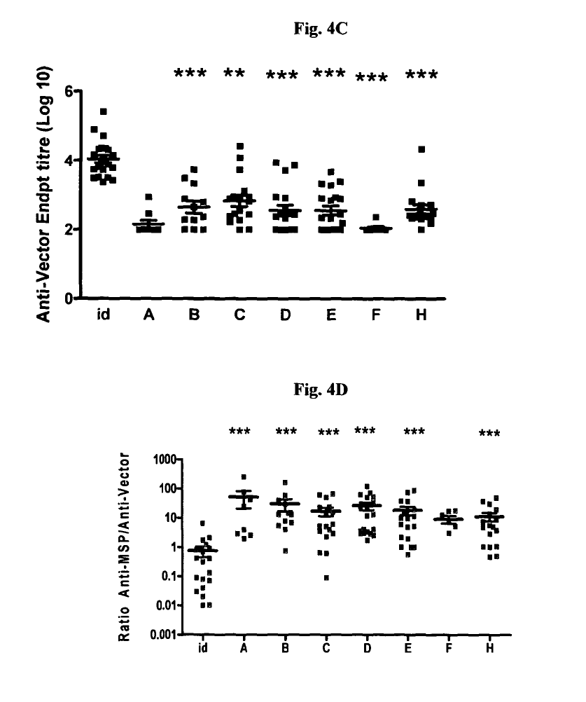

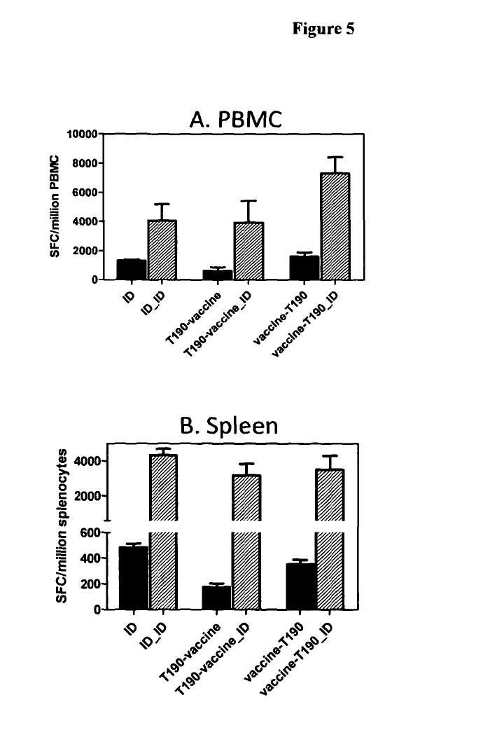

To determine if the design of a microneedle array can impact on the induction of antibody responses, we immunized C57BL/6 mice with a recombinant adenovirus virus vector that induces antibody responses to the encoded Plasmodium yoelii antigen MSP-1 (Draper et al (2009) Cell Host Microbe 5, 95-105 and (2008) Nat Med 14, 819-21). Similar to the induction of T cell responses, vaccine administration using low pore volume microneedle arrays resulted in a lower antibody titre after immunization compared to ID administration. Vaccination with microneedle arrays that generated intermediate pore volumes resulted in slightly increased antibody titres compared to ID and small arrays (FIG. 4A). However, when all mice were boosted by the ID route with MVA-MSP1, a significant increase in antibody titre was observed in mice that were primed with microneedle arrays that produced intermediate or large pore volumes compared to ID immunization (FIG. 4B). Induction of antibody responses to the virus vector encoding the antigen of interest is a significant problem in the field. However, priming with microneedle arrays that possess small, intermediate or large volumes induced a significantly (*** p<0.001, ** p<0.01) decreased antibody response to the adenovirus vector (as measured by ELISA) compared to intradermal administration. Specifically, when the ratio of the anti-antigen to anti-vector response is examined 50 days after the priming immunization, it was determined that the anti-antigen response was 10 to 100-fold higher when microneedle arrays were used to deliver the vaccine compared to intradermal immunization. This significantly greater frequency of antibody responses to the antigen compared to the vector is favourable for homologous prime-boost with adenovirus.

### Example 4

### Investigating the Optimal Order of Vaccine and Array Application

In a series of studies, it was discovered that applying the vaccine first and then the array induces a stronger immune response compared to administering the vaccine subsequent to applying a microneedle array (FIG. 5). Mice were immunised with MVA-PbCSP intradermally (id) or using an intermediate pore volume microneedle array, termed ‘T190’ (total pore volume=0.0285 mm³). Pores were first created by applying the microneedle array to the skin and the vaccine was then administered to the treated area, termed ‘T190-vaccine’. Alternatively vaccine was first administered onto the skin to which the microneedle array was then applied; termed ‘vaccine-T190’. Fourteen days later animals were boosted with MVA-PbCSP by the id route. Antigen-specific CD8⁺ T cell responses in Peripheral Blood Mononuclear Cells (FIG. 5A) and spleen (FIG. 5B) were assessed by IFN-γ ELISPOT 12 days after the prime immunisation (filled bars) and 12 days after the boost (striped bars). Data is representative of three separate experiments, with four animals per group. It can be observed that creation of the pores first and subsequent application of the vaccine (T190-vaccine) induces a weaker T cell response in peripheral blood post-prime and in the spleen compared to application of vaccine and subsequent administration of the microneedle array (vaccine-T190).

### Example 5

### Percutaneous Vaccine Delivery in Pig Skin

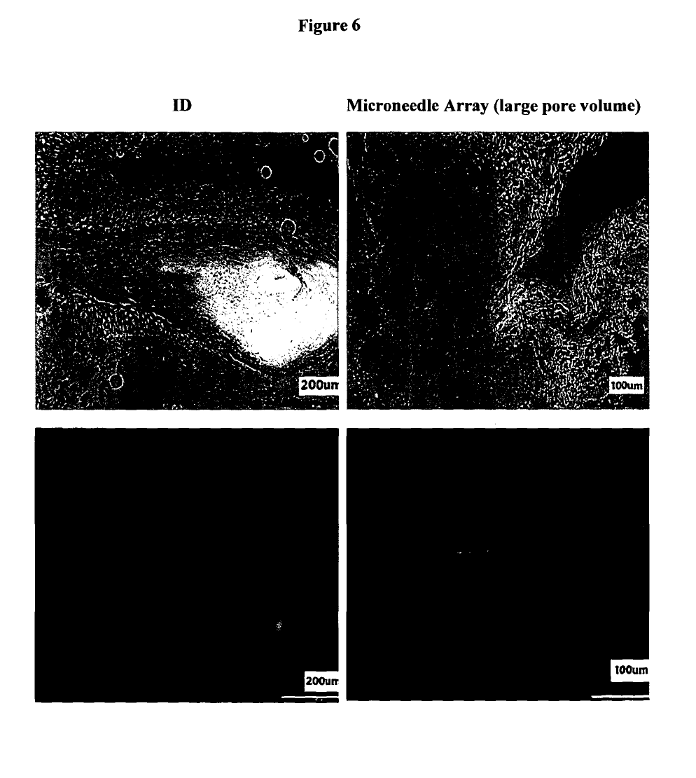

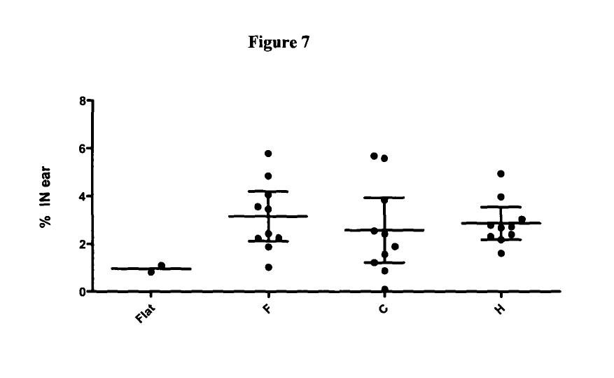

The pig has long been recognised as a preferred animal model for skin-based delivery studies. Swine skin is more similar to human skin in total thickness and hair follicle density than is mouse skin. In connection with the present invention, the pig model was used to verify the utility of the vaccination methods in humans (FIG. 6). Administration of vaccine (MVA-RFP) by the id route resulted in the expression of the RFP transgene in a concentrated, bolus around the site of needle insertion. In contrast, vaccine antigen was expressed in the epidermal area of the skin subsequent to microneedle array delivery.

### Example 6

### Determination of Dose Delivered

A key advantage to percutaneous vaccination is the reduction in the vaccine dose required to induce protective immunity. Based on the decreased fluorescence detected in pig skin when MVA expressing red fluorescent protein (MVA-RFP) is delivered by microneedle array compared to id (FIG. 6), it is expected that the microneedles detailed in this invention deliver a reduced dose of vaccine. It was determined, using red fluorescent beads (100 nm diameter), that the percentage of starting material that was delivered into the skin was between 0.5% and 5.8%. The dose delivered was independent of microneedle array design (FIG. 7).

### Methods

### Microneedle Array Design

Silicon microneedles were fabricated using wet-etch technology as described in US2007/0134829A1. The area of each microneedle array and the length and number of microneedles per array were designed to produce a microneedle array that created specific total pore volumes when inserted into skin. Each microneedle array in the examples was either 5.4 mm×5.4 mm or 7.4×7.4 mm in size. The height of the microneedles ranged from 100 μm to 300 μm and the number of microneedles per array ranged from 16 to 100. As examples, microneedle array type A was designed to posses 16 needles per array, where each needle was 100 μm in height and the area of each array was 5.4×5.4 mm. The total pore surface area and pore volume created by each array is detailed in Table 1.

### Vaccines

The construction, design and preparation of Modified Vaccinia Virus Ankara (MVA) expressing P. berghei CSP (MVA-PbCSP), red fluorescent protein (MVA-RFP) and Adenovirus (AdHu5) and MVA expressing the 42 kD version of Plasmodium yoelii merozoite surface antigen (MSP42) have been previously described (Draper et al (2008) and (2009) as above; Hutchings et al (2005) J Immunol 175, 599-606; Gilbert, et al (2002) Vaccine 20, 1039-45). All viruses were resuspended in endotoxin-free PBS for immunization.

### Immunogenicity Studies

Female BALB/c or C57BL/6 mice 4-6 weeks old (Harlan UK) were used in all experiments. Mice were immunized with 1×10⁶pfu MVA or 1×10¹⁰vp adenovirus. Vaccine was administered with a conventional 28G needle and syringe intradermally (id) into the ear. Alternatively, 5 μl of vaccine was placed on the dorsal surface of the ear and administered to the mouse by pressing a microneedle array onto the ear, using moderate thumb pressure, approximately 10-35N. Mice were primed on day 0. Post-prime T cell responses were analysed in the spleen and lymph node on day 14 after MVA immunization and day 55 after adenovirus immunization. Mice were boosted by the id route or using a microneedle array at day 14 post-MVA or day 56 post-Adenovirus immunization. Vaccine-induced immunity was tested in all groups 14 days after boosting.

Antigen-specific T cell responses were analysed by intracellular cytokine staining and flow cytometry (ICS) or IFN-γ ELISPOT. Flow cytometric analyses were performed using an LSRII (BD Biosciences) and data were analyzed with FlowJo (Tree Star) software. Analysis of multifunctional T cell responses was performed by using Boolean analysis in FlowJo software and SPICE 4.0 (M. Roederer NIH, Bethesda). Three major subsets of Ag-experienced T cells were defined according to their expression of CD62L and CD127. These markers are associated with central memory T cells (T_{CM}: CD62L⁺ CD127⁺), effector memory T cells (T_{EM}: CD162L⁻ CD127⁺), and effector T cells (T_{E}: CD62L⁻ CD127⁻). The ELISPOT assay was performed as previously described (Moore et al., J. Immunology 2005 175, 7264).

Antigen-specific antibody responses were assessed in serum that was collected from tail vein blood samples. Blood was allowed to clot overnight at 4° C. then centrifuged at 12000 rpm for 3 minutes and the serum collected and stored at −20° C. Individual mouse serum was analysed for anti-MSP42 antibodies by an indirect ELISA (Draper et al (2008) and (2009) as above)

### Ex Vivo Assessment of Vaccine Delivery

Pig skin was setup in a short-term ex vivo culture (Coulman et al., Current Drug Delivery, 2006, 3, 65-75). MVA-RFP was injected intradermally or administered using a microneedle array and skin was cultured for 14 hours at 37° C. to permit virus infection and transgene expression. Skin was then snap frozen and cryo-sectioned into 10 μm sections. Samples were examined by light microscopy (40×) or were stained with Hoechst33342 to identify cell nuclei and examined using a fluorescence microscope (20×).

TABLE 1
Examples of Needles

| Micro- | Micro- | Number of | Array | Total | Pore |
|---|---|---|---|---|---|
| needle | needle | Microneedles | Area | pore | volume |
| Array | height | per Array | (mm²) | volume mm³ | range |
| A | 100 μm | 16 | 29.16 | 0.0018 | Small |
| F | 100 μm | 100 | 29.16 | 0.0113 | Small |
| B | 200 μm | 16 | 29.16 | 0.0145 | Intermediate |
| D | 200 μm | 25 | 29.16 | 0.0227 | Intermediate |
| E | 200 μm | 36 | 29.16 | 0.0326 | Intermediate |
| C | 300 μm | 16 | 29.16 | 0.0499 | Intermediate |
| G | 200 μm | 81 | 54.76 | 0.0734 | Large |
| T125 | 125 μm | 400 | 100 | 0.0902 | Large |
| H | 300 μm | 36 | 54.76 | 0.1123 | Large |

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A prime-boost vaccination method which comprises:
(i) administering a priming vaccine using a microneedle array having a small total microneedle volume; and
(ii) administering a boosting vaccine using a microneedle array having a large total microneedle volume.

2. The prime-boost vaccination method according to claim 1, wherein the large total microneedle volume is at least two-fold greater than the small total microneedle volume.

3. The prime-boost vaccination method according to claim 1, wherein the small total microneedle volume is between 0.0005 and 0.014 mm³.

4. The prime-boost vaccination method according to claim 3, wherein the large total microneedle volume is at least 0.05 mm³.

5. The prime-boost vaccination method according to claim 3, wherein the large total microneedle volume is between 0.07 and 0.25 mm³.

6. A kit comprising:
(i) a microneedle array suitable for administering a priming vaccine having a small total microneedle volume; and
(ii) a microneedle array suitable for administering a boosting vaccine having a large total microneedle volume.

7. The kit according to claim 6, wherein the microneedle array suitable for administering a priming vaccine has a total microneedle volume of between 0.0005 and 0.014 mm³; and/or the microneedle array suitable for administering a boosting vaccine has a total microneedle volume of at least 0.05 mm³.

8-10. (canceled)

11. The method according to claim 1, wherein the priming vaccine and boosting vaccine are homologous.

12. The method according to claim 1, wherein the priming vaccine and the boosting vaccine comprises a modified vaccinia virus Ankara (MVA) vector expressing an antigen or epitope of interest.

13-14. (canceled)

15. The kit according to claim 6, which also comprises a priming vaccine composition.

16-38. (canceled)

39. The method according to claim 1, wherein the microneedle array is fabricated from silicon.

40. The method according to claim 1, wherein the microneedle array is fabricated from a polymer.
